(19) United States
(12) Patent Application Publication
(10) Pub. No.: US 2009/0043276 A1
(43) Pub. Date: Feb. 12, 2009

(54) DRUG DELIVERY DEVICE, COMPOSITIONS AND METHODS RELATING THERETO

(75) Inventor: Jan Weber, Maastricht (NL)

Correspondence Address: VIDAS, ARRETT & STEINKRAUS, P.A., SUITE 400, 6640 SHADY OAK ROAD, EDEN PRAIRIE, MN, 55344, US

(73) Assignee: BOSTON SCIENTIFIC SCIMED, INC., Maple Grove, MN (US)

(21) Appl. No.: 11/836,237

(22) Filed: Aug. 9, 2007

(51) Int. Cl. A61M 31/00; A61F 2/00; A61M 25/00

(52) U.S. Cl. 604/502; 424/426; 604/57

(57) ABSTRACT

An implantable drug delivery device including at least one flexible elongate element, the flexible elongate element including a polymeric carrier for a drug and anchors for attachment to a vessel wall of the patient.

### FIELD OF THE INVENTION

The present invention relates to the field of therapeutic drug delivery, and in particular to devices employed for therapeutic drug delivery to specific target sites within the body of a patient, and methods of using the same.

### BACKGROUND OF THE INVENTION

Systemic delivery of drugs for various diseases has been relatively effective. However, the controlled, localized delivery of drugs to a target site or region in the body of a patient has become increasingly desirable because higher doses can be maintained locally and the delivery of drugs directly to diseases tissue can be sustained over a longer period of time which in turn can minimize side effects.

Contemporary therapeutic delivery techniques include hypodermic needle injections performed outside of the body, coating expandable medical balloons used for expanding occluded vessels within a patient, and placing implantable intraluminal devices such as stents within the body of a patient.

Research has been increasingly centered about improved devices and methods for the controlled, local delivery of therapeutic agents which can be sustained over periods of time within the body of a patient.

### SUMMARY OF THE INVENTION

The present invention relates to an implantable medical device for controlled local delivery of therapeutic substances over extended periods of time, to systems for delivery thereof, and to methods and compositions for use therewith.

In one aspect, the present invention relates to an implantable drug delivery device including at least one flexible elongate element including a polymeric carrier for a therapeutic agent or mixture of therapeutic agents, and anchors for attachment to a vessel wall of the patient. In some embodiments, the polymeric carrier may control the rate of drug release.

The carrier may be the flexible elongate element itself, may be in the form of a coating on the flexible elongate element, or may be in the form of microparticles disposed on the flexible elongate element.

In another aspect, the present invention relates to a catheter assembly for delivering an insertable or implantable drug delivery device to a treatment site in a vessel of a patient, the catheter including an elongate catheter shaft having an inner surface defining a lumen, a flexible elongate drug delivery device disposed in the lumen and an elongate deployment device releasably secured to the flexible elongate drug delivery device. The flexible elongate drug delivery includes a carrier of a therapeutic agent and anchors for securing the drug delivery device to a vessel wall. The elongate deployment device has a preset expanded configuration. Upon deployment in a vessel, the elongate deployment device assumes the preset expanded configuration and releases the elongate drug delivery device.

In one embodiment, the elongate deployment device is formed from a shape memory material such as a shape memory metal or metal alloy.

In another aspect, the present invention relates to a method including inserting or implanting a flexible elongate medical device into a vessel of a patient, the elongate medical device including a carrier of a therapeutic agent or a mixture of therapeutic agents and anchors for securing the flexible elongate medical device to a wall of the vessel and securing the elongate medical device to the vessel wall.

In one embodiment, the method includes providing a delivery catheter including an elongate flexible catheter shaft having an inner surface defining a lumen and having a distal portion, providing a flexible elongate drug delivery device within the distal portion of the lumen of the catheter shaft, the flexible elongate drug delivery device including a carrier of a therapeutic agent or mixtures of therapeutic agents and anchors for securing the drug delivery device to a wall of the vessel, positioning the distal portion of the delivery catheter at a treatment site in the vessel and deploying the flexible elongate drug delivery device from the catheter shaft and into the vessel, wherein the anchors secure the flexible elongate drug delivery device to the vessel wall.

In one aspect, the elongate drug delivery device is releasably secured to an elongate deployment device having a preset expanded configuration, wherein the elongate drug delivery device is released into the vessel when the elongate deployment device assumes its preset expanded configuration.

These and other aspects, embodiments and advantages of the present invention will become immediately apparent to those of ordinary skill in the art upon review of the Detailed Description and Claims to follow.

### BRIEF DESCRIPTION OF THE DRAWINGS

FIG. 1 illustrates one embodiment of a drug delivery device including a flexible elongate element loaded with therapeutic agent(s).

FIG. 2 illustrates an embodiment of a drug delivery device including a flexible elongate element having a coating disposed thereon, the coating including a therapeutic agent(s).

FIG. 3 is a radial cross-section of a drug delivery device taken at section 3-3 in FIG. 2.

FIG. 4 illustrates an embodiment of a drug delivery device including a flexible elongate element having microparticles disposed thereon.

FIG. 5 is perspective view of the distal end of an embodiment of a delivery catheter for use in combination with one embodiment of a drug delivery device as disclosed herein.

FIG. 6 is a radial cross-section taken at section 6-6 in FIG. 5.

FIG. 7 is a radial cross-section of a shape memory metallic wire.

FIGS. 8-9 are perspective views of the distal end of an embodiment of a delivery catheter similar to that shown in FIG. 5 illustrating various stages of deployment of one embodiment of a drug delivery device as disclosed herein.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

All US patents and applications and all other published documents mentioned anywhere in this application are incorporated herein by reference in their entirety. Any copending patent applications, mentioned anywhere in this application are also hereby expressly incorporated herein by reference in their entirety

For the purposes of this disclosure, like reference numerals in the figures shall refer to like features unless otherwise indicated.

The present invention relates to a drug-delivery device for controlled, local delivery of drugs, the device including a flexible elongate element, the flexible elongate element including a carrier for a therapeutic agent and anchors for securing the elongate flexible element to the vessel wall of a patient, and to methods of making and using the same.

Any of a variety of techniques may be employed for loading the flexible elongate element with therapeutic agents including forming the flexible elongate element itself from a polymeric composition which functions as the carrier for the therapeutic agent(s), coating the flexible elongate element with a polymer composition that functions as the carrier for the therapeutic agent(s), or securing polymeric microparticles to the flexible elongate element wherein the microparticles function as the carrier for the therapeutic agent(s).

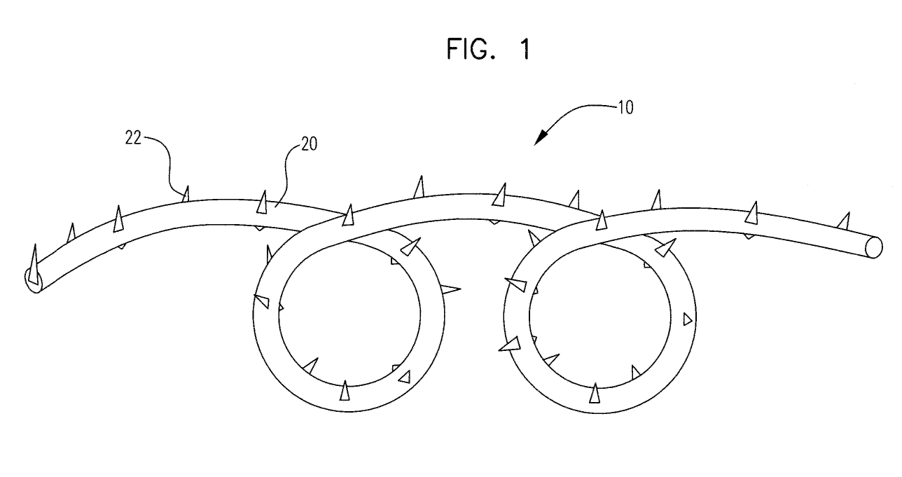

Turning now to the figures, FIG. 1 illustrates generally at 10, one embodiment of a drug-delivery device according to the present invention Device 10 includes a flexible elongate element 20. Flexible elongate element 20 may be comprised of a single filament, fiber or thread, or may be two or more filaments, fibers or threads. The threads may be intertwined using any method known in the art including twisting, braiding, weaving, roving, etc. As used herein, the terms string, filament, fiber, thread, cord, etc. shall be used interchangeably. In this embodiment, flexible elongate element 20 is shown equipped with anchors 24 for securing flexible elongate element 20 to a vessel wall (not shown). The anchors 24 may be nails, hooks, tacks, pins, or the like, which can be driven into a vessel wall upon deployment of the device. Such nails, hooks, tacks, pins of the like may be pushed through the flexible elongate element. The device, in this embodiment, may be referred to as a “punaise string” (punaise is French for tack).

The flexible elongate element may have a diameter size from about 20 microns to about 150 microns.

Suitably, the anchors 24 are formed from a biocompatible material such as a bioerodible metal, polymer, etc. Any suitable biocompatible metal may be employed including, but not limited to, magnesium and magnesium alloys, iron, sodium sulfate, tungsten, etc.

Biocompatible adhesive materials may also be employed. For example, cyanoacrylate exhibits good adhesion to human tissue and may be employed. Another example is to apply a biotinylated-Sialyl Lewis^{X} (sLe^{X}) to the elongate flexible element. Biotinylated-Sialyl sLe^{X} will stick to the endothelial layer at an inflammatory site. See Characterization of biodegradable drug delivery vehicles with the adhesive properties of leukocytes II: effect of degradation on targeting activity, Eniola, A. Omolola and Hammer, Daniel A., 26 Biomaterials, pp. 661-670 (2005). Drug-loaded poly(lactic-co-glycolic acid) (PLGA) microspheres were coated with biotinylated-Sialyl Lewis^{X} (sLe^{X}), a carbohydrate that serves as a ligand to selecting, and mimic the behavior of leukocytes on selectins in flow chambers.

An adhesive material may be employed, with or without tacks, nails, pins or other anchoring devices. A self-expanding tubular member as described herein may be equipped with voids or holes through which an adhesive material mixed with therapeutic agent(s) is injected. In this embodiment, the therapeutic agent(s) may preferably be encapsulated within a polymeric microparticle.

Suitably, flexible elongate element 20 is formed from materials that are biocompatible, and bioresorbable/biodegradable, including both polymer materials and metals, the coating degrading in an aqueous environment such as within the body of a patient. Degradation may occur through any of a variety of mechanisms including hydrolysis, weakening of ionic bonds, hydrogen bonds or Van der Waals forces, or other dissolution mechanisms. Suitably, the flexible elongate element is formed from a flexible material so that when inserted into a vessel it can easily follow the contour of the vessel wall.

Alternatively, a material that may be delivered while in a flexible state, and then cured to a harder, more rigid polymer material may also be employed. For example, a thermosetting polymer composition such as an ultraviolet (UV) curable polymer composition may be employed. An optical fiber may then be delivered through a hollow, flexible elongate element 20 which is in its expanded state and employed to cure the polymer composition. UV curable monomers, oligomers and polymers may be employed in the curable compositions. One example of a UV curable polymer compositions having bioadhesive properties include, but are not limited to, copolymers of N-vinyl pyrrolidone with 2-acrylamido methyl 1-propane sulfonic acid, vinyl succinimide, glycidyl acrylate, and 2-isocyanatoethyl methacrylate. See Kao, F J, et al., UV curable bioadhesives: copolymers of N-vinyl pyrrolidone, J. Biomed. Mater. Res., 38(3), pgs 191-196 (Fall, 1997). Other examples include, but are not limited to, UV curable epoxies, acrylamides, acrylates, urethanes and polyurethane oligomer compositions, aliphatic urethane acrylate oligomers, and UV curable silicones such as epoxy functional polysiloxanes. See for example U.S. Patent Publication No. 2006/0153892. See also U.S. Pat. No. 5,591,199 for UV curable polymers compositions, the entire content of which is incorporated by reference herein.

Other thermosetting polymer compositions which may be delivered in a flexible, uncured state, and then cured into a harder, more rigid polymer material include moisture curable polymer compositions which cure upon exposure to an aqueous environment. Examples include, but are not limited to organo siloxane polymers (U.S. Pat. No. 6,406,792 which is incorporated by reference herein in its entirety) and urethanes or polyurethanes oligomer compositions and mixtures thereof.

See also commonly assigned U.S. Pat. No. 5,725,568 for both biocompatible moisture curable polymer compositions and UV curable polymer compositions, the entire content of which is incorporated by reference herein.

Degradable polymer materials may be employed in forming flexible elongate element 20. Examples of suitable polymer materials include, but are not limited to, polyhydroxyalkanoates such as poly(hydroxybutyrate) (PHB), poly(hydroxyvalerate) (PHV) and poly(hydroxybutyrate-co-valerate), polylactones such as polycapolactone (PCL), poly(L-lactic acid) (PLA), poly(glycolic acid) (PGA), poly(D,L-lactic acid), poly(lactide-co-glycolide) (PLGA), polydioxanone, polyorthoesters, polyanhydrides, poly(glycolic acid-co-trimethylene carbonate), polyphosphoesters, polyphosphoester urethanes, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen, hyaluronic acid, etc., and mixtures thereof. Bioabsorable polymers are disclosed in U.S. Pat. No. 6,790,228, the entire content of which is incorporated by reference herein.

Hydrophilic polymer materials may also be employed. As used herein, the term “hydrophilic” is used to refer to water having various degrees or water sensitivity including those materials that are water soluble, dispersible, dissolvable, etc. As used herein, the term “water soluble” shall include those materials which have partial solubility in water.

Suitable hydrophilic polymers include those that have non-crosslinked structures having hydrophilic groups thereon, such as —OH, —COOH, —CONH, —COO—, etc. The hydrophilicity of the polymer can be controlled by the number of such groups, as well as the polymer structure.

Examples of hydrophilic polymers include, but are not limited to, polyalkylene glycols such as polyethylene glycol (PEG) and modified polyethylene glycols, polyethylene oxide and hydrophilic block copolymers of polyethylene oxide and polypropylene oxide, carbohydrates, sugar alcohols such as mannitol, polyols, monosaccharides, oligosaccharides, polysaccharides and modified polysaccharides such as Heparin (mucopolysaccharide), hydrophilic polyurethanes such as polyether aliphatic polyurethanes, hydrophilic polyamides, hydroxyethyl methacrylate (HEMA), salts of polyacrylic acid such as the alkali metal salts (Na, K are the most common) or alkaline earth metal salts of polyacrylic acid, polyvinyl alcohol, polyvinyl acetate, polyvinylpyrrolidone (a hydrophilic poly(N-vinyl lactam), cellulose and hydrophilic modifications thereof such as carboxymethyl cellulose, methyl cellulose, hydroxyethyl cellulose and hydroxypropyl cellulose, methyl vinyl ether-maleic anhydride copolymers, etc. For hydrophilic polymer materials, see for example, U.S. Pat. No. 5,509,899 and U.S. Patent Publication No. 2006/0212106, each of which is incorporated by reference herein in its entirety.

In the embodiment shown in FIG. 1, the flexible elongate element 20 functions as the carrier for the therapeutic agent(s).

Any therapeutic agent may be employed herein. As used herein, the terms, “therapeutic agent”, “drug”, “pharmaceutically active agent”, “pharmaceutically active material”, “beneficial agent”, “bioactive agent”, and other related terms may be used interchangeably herein and include genetic therapeutic agents, non-genetic therapeutic agents and cells. A drug may be used singly or in combination with other drugs. Drugs include genetic materials, non-genetic materials, and cells.

Some exemplary drugs include, but are not limited to, anti-restenosis drugs, such as paclitaxel, sirolimus, everolimus, tacrolimus, dexamethoasone, estradiol, ABT-578 (Abbott Laboratories), trapidil, liprostin, Actinomycin D, Resten-NG, Ap-17, clopidogrel and Ridogrel.

Therapeutic agents are disclosed in commonly assigned copending U.S. Patent Publication Nos. 2004/0215169 and 2006/0129727, each of which is incorporated by reference herein in its entirety.

The flexible elongate element 20 may be loaded with the therapeutic agent(s) using any suitable method known in the art including during melt extrusion. Some drugs, however, are heat sensitive so the heat history must be controlled, or alternative methods employed. For example, paclitaxel may exhibit degeneration during melt extrusion that can inhibit its effectiveness.

Other methods include application of the therapeutic agent(s) out of a solvent such as by spraying, dipping, painting and so forth.

In yet another embodiment, the flexible elongate element 20 may be in the form of a hollow tubular member, wherein the tubular member is filled with the therapeutic agents(s), for example, using a gel mixed with the therapeutic agent(s). The wall or the tubular member may include voids or holes for exposure of the interior to bodily fluids.

An alternative method for heat sensitive therapeutic agent(s) may be to spin flexible elongate element 20 from a solvent solution including polymer and therapeutic agent(s) using conventional wet spinning techniques or through a process referred to in the art as electrospinning. However, electrospinning is employed to produce submicron size fibers. Therefore, this technique may be more suitable for those flexible elongate elements including a plurality of fibers which are braided or woven together.

Conventional wet or gel spinning methods may be employed to produce larger, micro-sized fibers, for example.

In some preferred embodiments, the polymer employed is a biodegradable/bioresorbable material and includes l-lactide, d,l-lactide, glycolide, or a combination thereof. Examples include, but are not limited to, polylactide and polylactide-co-glycolide.

The drug release kinetics are controlled by the type of degradable polymer matrix employed, the number of layers, the wall thickness, and by the drug itself, which can, for example, have a different dissolution rate in an amorphous state as opposed to a crystalline state.

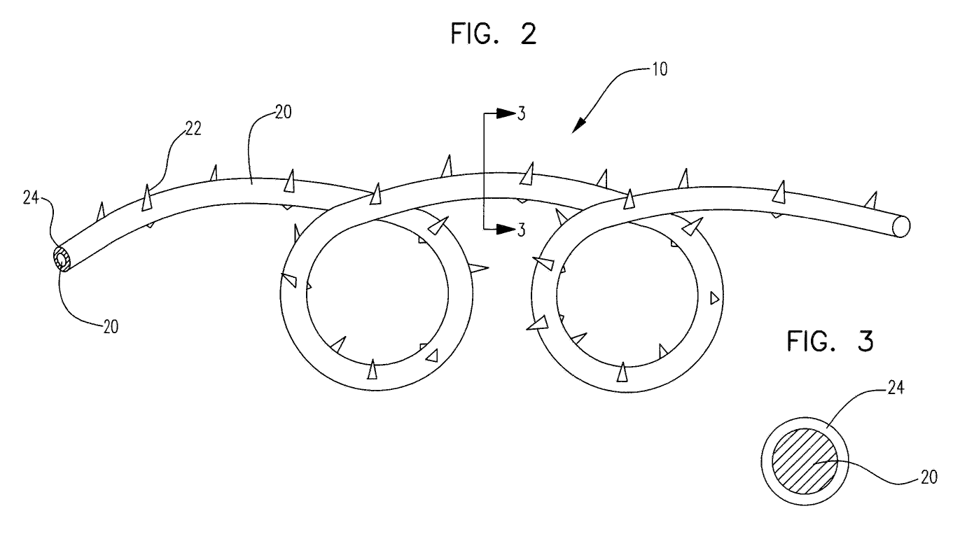

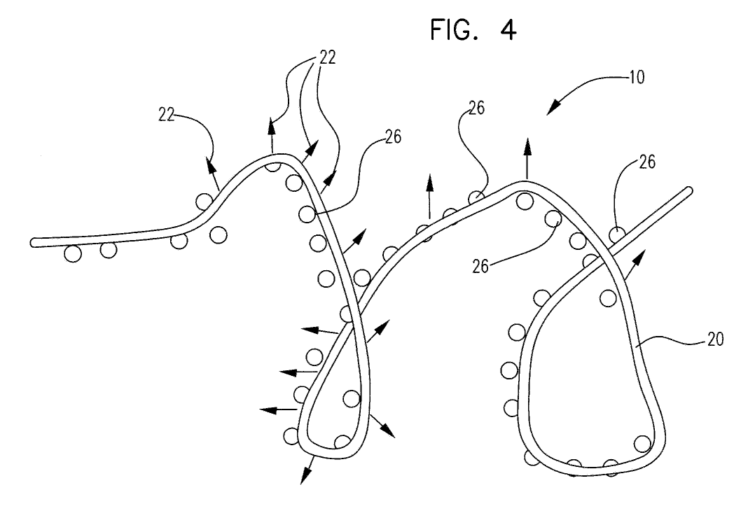

FIG. 2 illustrates another embodiment of a drug delivery device 10 including a flexible elongate element 20 including anchors 22 in the form of tacks, pins, hooks or the like and having a polymeric coating 24 disposed thereon. Polymeric coating 24 is a carrier for the therapeutic agent(s). FIG. 3 is a radial cross-section taken at section 3-3 in FIG. 2 showing coating 24 disposed on flexible elongate element 20.

In this embodiment, flexible elongate element 20 may be formed from a polymer material as discussed above, or flexible elongate element 20 may be in the form of a very fine metallic wire formed from a flexible, biocompatible, and dissolvable and/or bioresorbable metal or metal-based composition Suitable metal or metal-based compositions include, but are not limited to, iron or magnesium wire, tungsten, alloys of iron and silver, sodium sulfate, etc.

These wires can also be in the form of a single strand, or may be formed from two or more braided or woven strands, for example.

Coating 24 may include any polymer material suitable for the formation of a drug-eluting coating. Desirably, the coating is biocompatible, and bioresorbable/biodegradable. Any of the polymer materials discussed for formation of the flexible elongate element 20, above, may also be employed in the drug-eluting coating 24. Preferred coating materials include, but are not limited to, those polymers formed using l-lactide, d,l-lactide or glycolide, for example, polylactide and polylactide-co-glycolide.

Any suitable coating method may be employed. In one embodiment, polymer and therapeutic agent(s) are mixed together in a solvent and applied to the flexible elongate element by brushing, dipping, spraying, or pulling the flexible elongate element through a solvent bath containing the polymer and therapeutic agent(s).

For application of a drug-eluting coating see commonly assigned U.S. Patent Publication No. 2001/0032014, the entire content of which is incorporated by reference herein.

FIG. 3 illustrates an embodiment wherein the carrier for the therapeutic agent(s) is in the form of microparticles 26 which are individually attached to the flexible elongate element 20.

As used herein, the term “microparticle” shall include particle sizes of about 0.05 microns (50 nanometers) up to about 25 microns (25,000 nm), and suitably up to about 20 microns (20,000 nm).

The microparticle shell may be formed from any suitable biocompatible polymer composition. Suitably, the polymer composition includes a bioerodible/biodegradable polymer material, or a hydrophilic polymer material as discussed which is discussed above. Any of the same materials employed for the formation of flexible elongate element 20 may be employed in forming the microparticle shell as well. The microparticle shell may the same composition or a different composition than flexible elongate element 20.

The microparticle shell may be attached to the surface of the flexible elongate element using a variety of methods.

One method is to form both the elongate flexible filament and the microparticle of the same polymer material, and to employ a suitable method such as solvent or heat to meld them together.

Another method is to employ a layer-by-layer (LbL) self-assembly techniques for both the preparation of the microparticle itself, and for securement of the microparticle 26 flexible elongate element 20. Using LbL self-assembly techniques, the sequential absorption of oppositely charged species from solution, e.g. aqueous media, can be employed to prepare multi-layer films. The charge on the outer layer is reversed upon deposition of each subsequent polyelectrolyte layer.

Formation of the particle, itself using LbL techniques typically involves coating charged particles, which are dispersed in aqueous media, via nanoscale, electrostatic, self-assembly using charged polymeric (polyelectrolyte) materials. The charged particles can serve as templates for the polyelectrolyte layers. The charge on the outer layer is reversed upon deposition of each sequential polyelectrolyte layer. This stepwise deposition of subsequent polyelectrolyte layers results in multilayer shells that are known to provide controlled drug release. Shell properties such as the number of layers, wall thickness, and permeability can be tuned to provide an appropriate drug release profile.

Any material which can either be provided with a surface charge, or one inherently having a surface charge, such as a protein, may be employed as a template for formation of the microparticle. Examples of charged polymeric therapeutic agents include polynucleotides (e.g., DNA and RNA) and polypeptides.

Uncharged materials can also be encapsulated using LbL techniques using a variety of different methods including, for example, (a) providing the compound in finely divided form using, for instance, (i) colloid milling or jet milling or precipitation techniques, to provide solid particles, or (ii) emulsion technique to provide liquid particles within a continuous liquid or gel phase. The particles can be provided with a surface charge, for example, by providing least one amphiphilic substance (e.g., an ionic surfactant, an amphiphilic polyelectrolyte or polyelectrolyte complex, or a charged copolymer of hydrophilic monomers and hydrophobic monomers) at the phase boundary between the solid/liquid template particles and the continuous phase (typically an aqueous phase).

Amphiphilic compounds include any that have both hydrophilic and hydrophobic groups. Suitable amphiphilic compounds for use in formation of a microparticle shell using LbL techniques should also have at least one electrically charged group, i.e. ionic amphiphilic compounds, in order to function as a template particle for deposition of subsequent layers. The amphiphilic compound may be an amphiphilic polyelectrolyte, for example, poly(styrene sulfonate) (PSS) wherein the hydrophobic group is aromatic, i.e. styrene.

Other examples of suitable amphiphilic compounds include cationic and anionic surfactants including, but not limited to,

Cationic and anionic surfactants can also be used as amphiphilic substances. Cationic surfactants include quaternary ammonium salts (NR₄⁺X⁻), for example, didodecyldimethylammonium bromide (DDDAB), alkyltrimethylammonium bromides such as hexadecyltrimethylammonium bromide (HDTAB), dodecyltrimethylammonium bromide (DTMAB), myristyltrimethylammonium bromide (MTMAB), or palmityl trimethylammonium bromide, or N-alkylpyridinium salts, or tertiary amines (NHR₃⁺X⁻), for example, cholesteryl-3β-N-(dimethyl-aminoethyl) carbamate or mixtures thereof, wherein X⁻ is a counteranion, e.g. a halide.

Anionic surfactants include alkyl or olefin sulfate (ROSO₃ M), for example, a dodecyl sulfate such as sodium dodecyl sulfate (SDS), a lauryl sulfate such as sodium lauryl sulfate (SLS), or an alkyl or olefin sulfonate (RSO₃M), for example, sodium-n-dodecyl-benzene sulfonate, or fatty acids (RCOOM), for example, dodecanoic acid sodium salt, or phosphoric acids or cholic acids or fluoro-organics, for example, lithium-3-[2-(perfluoroalkyl)ethyl-thio] propionate or mixtures thereof, where R is an organic radical and M is a countercation.

Once a charged template particle is provided, it can be coated with a layer of an oppositely charged polyelectrolyte. Multilayers are formed by repeated treatment with oppositely charged polyelectrolytes, i.e., by alternate treatment with cationic and anionic polyelectrolytes. The polymer layers self-assemble onto the pre-charged solid/liquid particles by means of electrostatic, layer-by-layer deposition, thus forming a multilayered polymeric shell around the cores.

Suitable materials for use in formation of the polymeric shell include, but are not limited to, ionic polymers including polyelectrolytes (contain cationic or anionic groups) and polyzwitterions (contain both anionic and cationic groups), proteins, ribonucleotides including RNA and DNA, inorganic particles, lipids, etc.

A polyelectrolyte is a macromolecular substance which, on dissolving in water or another ionizing solvent, dissociates to give polyions (polycations or polyanions). Some may also produce ions of small and opposite charge. Polyelectrolytes include polyacids, polybases, polysalts or polyampholytes or polyzwitterions which include both cationic (polycation) and anionic repeat groups (polyanion). The polyelectrolytes employed herein may be synthetic, semi-synthetic or naturally occurring (e.g. proteins and polysaccharides).

There is no limitation as to the polyelectrolyte which may be employed herein providing that the molecules used have sufficiently high charge and/or are capable of binding with the layer beneath via other kinds of interactions such as hydrogen bonds or some other type of interaction. Polyelectrolytes may be anionic or cationic in nature and include but are not limited to carboxylic, sulfate, and amine functionalized polymers. Examples of polyacids include, but are not limited to, poly(meth)acrylic acids, polyphosphoric acids, polyvinylsulfonic acids, polyvinylsulfuric acids, polyvinylphosphonic acids, etc. Corresponding salts, i.e. polysalts, include, but are not limited to, polyphosphates, polyvinylsulfates, polyvinylsulfonates, polyvinylphosphonates and poly(meth)acrylates.

Polybases contain groups which are capable of accepting protons, e.g., by reaction with acids, with a salt being formed. Examples of polybases having dissociable groups within their backbone and/or side groups are polyallylamine, polyethylimine, polyethylene amine, polyvinylamine and polyvinylpyridine. By accepting protons, polybases form polycations.

Examples of biopolymers include, but are not limited to, alginic acid, hyaluronic acid, gum arabicum, nucleic acids, pectins, proteins, heparin (mucopolysaccharide), chitosan, and chemically modified biopolymers such as carboxymethyl chitosan, carboxymethyl cellulose, carboxymethyl starch, carboxymethyl dextran, heparin sulfonate, chondroitin sulfate and lignin sulfonate.

Examples of anionic polyelectrolytes include, but are not limited to, polyacrylic acids and their salts, polymethacrylic acids and their salts, alginic acids and their salts, pectinic acids and their salts, carboxymethyl cellulose, hyaluronic acids and their salts, heparin, carboxymethyl starch, carboxymethyl dextran, heparin sulfate, chondroitin sulfate, poly(styrenesulfonate) polyanions (e.g., poly(sodium styrenesulfonate) (PSS)), eudragit polyanions, gelatin polyanions, carrageenan polyanions, etc.

Examples of cationic polymers include, but are not limited to, chitosan, cationic guar, cationic starch, polyethylene amine, protamine sulfate polycations, poly(allylamine) polycations (e.g., poly(allylamine hydrochloride) (PAH)), polydiallyldimethylammonium polycations, polyethyleneimine polycations, eudragit polycations, gelatine polycations, spermidine polycations, albumin polycations, etc.

Polyzwitterions or polyampholytes may be employed and include, but are not limited to, fibronectin, vitronectin, tenascin, elastin, laminin, gelatin, aggrecan, polysulfobetaines, etc.

See commonly assigned U.S. Patent Publication Nos. 20050129727 (Localized Drug Delivery Using Drug-Loaded Nanocapsules) and 2006/0212106 (Coatings for use on Medical Devices), each of which is incorporated by reference herein in its entirety. Each discusses in detail the use of LbL techniques, and the polyelectrolytes which may be employed for forming nanoparticles and coatings. See also U.S. Pat. No. 7,056,554 which also discusses the formation of polyelectrolyte capsules, also incorporated by reference herein in its entirety.

Preferably, the polyelectrolytes employed herein for formation of the microparticle shell are biocompatible and bioerodible/biodegradable in nature. Examples include, but are not limited to, polyglycolic acid (PGA), polylactic acid (PLA), polyamides, poly-2-hydroxy-butyrate (PHB), polycaprolactone (PCL) and poly(lactic-co-glycolic)acid (PLGA), protamine sulfate, polyallylamine, polydiallyldimethylammoniume, polyethyleneimine, chitosan, eudragit, gelatin, spermidine, albumin, polyacrylic acid, sodium alginate, polystyrene sulfonate, hyaluronic acid, carrageenin, chondroitin sulfate, carboxymethylcellulose, heparin, other polypeptides and proteins, and DNA, among others.

The template particle or core can be disintegrated. The desired therapeutic agent can be loaded into the shell using a variety of different techniques.

Some techniques take advantage of gradients across the capsule wall to effect precipitation or synthesis of a desired substance within the shell. For example, large macromolecules such as polymers cannot penetrate polyelectrolyte multilayers, while small solutes, for example, small molecule pharmaceuticals, can. Accordingly, the presence of macromolecules inside the capsules will lead to a difference in the physico-chemical properties between the bulk and the capsule interior, providing, for example, gradients in pH and/or polarity, which can be used to precipitate/synthesize materials within the capsules. Typically, a macromolecule is provided on the interior of the capsule by forming a double shell polyelectrolyte structure, after which the inner shell is decomposed.

Information on formation of the microparticles and loading the microparticles with therapeutic agents can be found in “A Novel Method for Encapsulation of Poorly Water-soluble Drugs: Precipitation in Polyelectrolyte Multilayer Shells,” I. L. Radtchenko et al., International Journal of Pharmaceutics, 242, 219-223 (2002), the disclosures of which is hereby incorporated by reference; in “Micron-Scale Hollow Polyelectrolyte Capsules with Nanosized Magnetic Fe₃O₄ Inside,” Materials Letters, D. G. Shchukin et al. (in press), the disclosure of which is hereby incorporated by reference; and in “Polyelectrolyte multilayer capsules as vehicles with tunable permeability,” Antipov, A. A. and Sukhorukov, G. B., Advances in Colloid and Interface Science, 111, 49-61 (2004), the disclosure of which is incorporated by reference herein.

For other methods of forming nanocapsules, see “Drug-loaded magnetic, hollow silica nanocomposites for nanomedicine,” W. Zhou et al., Nanomedicine: Nanotechnology, Biology, and Medicine 1, 233-237 (2005), the disclosure of which is incorporated by reference herein.

See also U.S. Pat. No. 7,056,554 (Production of Polyelectrolyte Capsules by Surface Precipitation), U.S. Patent Application No. 20020187197, WO 99/47252, WO 00/03797, WO 00/77281, WO 01/51196, WO 02/09864, WO 02/09865, WO 02/17888, “Fabrication of Micro Reaction Cages with Tailored Properties,” L. Dhne et al., J. Am. Chem. Soc., 123, 5431-5436 (2001), “Lipid Coating on Polyelectrolyte Surface Modified Colloidal Particles and Polyelectrolyte Capsules,” Moya et al., Macromolecules, 33, 4538-4544 (2000), “Microencapsulation of Organic Solvents in Polyelectrolyte Multilayer Micrometer-sized Shells,” S. Moya et al., Journal of Colloid and Interface Science, 216, 297-302 (1999); “Assembly of Alternated Multivalent Ion/Polyelectrolyte Layers on Colloidal Particles,” I. L. Radtchenko et al., Journal of Colloid and Interface Science, 230, 272-280 (2000); “Controlled Precipitation of Dyes into Hollow Polyelectrolyte Capsules,” G. Sukhorukov et al., Advanced Materials, Vol. 12, No. 2, 112-115 (2000), the disclosures of which are hereby incorporated by reference.

The wall thickness provided by the above layer-by-layer techniques will frequently range, for example, from about 1 nm to about 10,000 nm, depending on the type of polyelectrolytes used for the formation of the shell.

Using techniques such as those discussed above, a single drug can be encapsulated within a single microparticle, or two or more drugs can be encapsulated within a single microparticles. Moreover, two or more microparticles, each one holding a different drug, can be combined to provide for release of multiple drugs. If two or more drugs are encapsulated within a single microparticle, it may be desirable to have each drug within its own drug region in the case of adverse drug interactions, for example. For example, a first drug (e.g., a drug that addresses smooth muscle cell proliferation or inflammatory responses) can be provided in an inner region such as the core, an inner multilayer encapsulation can surround the core (e.g., to address drug interaction and/or delay diffusion), an additional layer containing a second drug (e.g., a drug that addresses acute arterial injury) can then be provided over the inner multilayer encapsulation, and an outer multilayer encapsulation can be provided over the layer containing the second drug. LbL techniques offer a wide variety of choices when selecting how the drugs are arranged within a microparticle.

The kinetic drug release rate can be controlled by the type of polymer employed in the shell of the microparticles or coating, by the wall thickness of the coating, the number of layers in the shell, and by the drug itself.

The LbL formed microparticles may be adhered to the flexible elongate element 20 using LbL techniques as well. For example, flexible elongate element 20 may be charged with a strong anionic or cationic coating, for example polyethylene imine (PEI) which is a polycation. PEI can be readily applied to flexible elongate element 20 using any suitable method such as dissolving the PEI in solvent and then dipping, spraying, brushing, running flexible elongate element 20 through a bath containing PEI, etc. The outermost layer of the LbL formed microparticle suitably has a corresponding negative charge, i.e. is polyanionic. For example, a polyacrylic acid carries a negative charge.

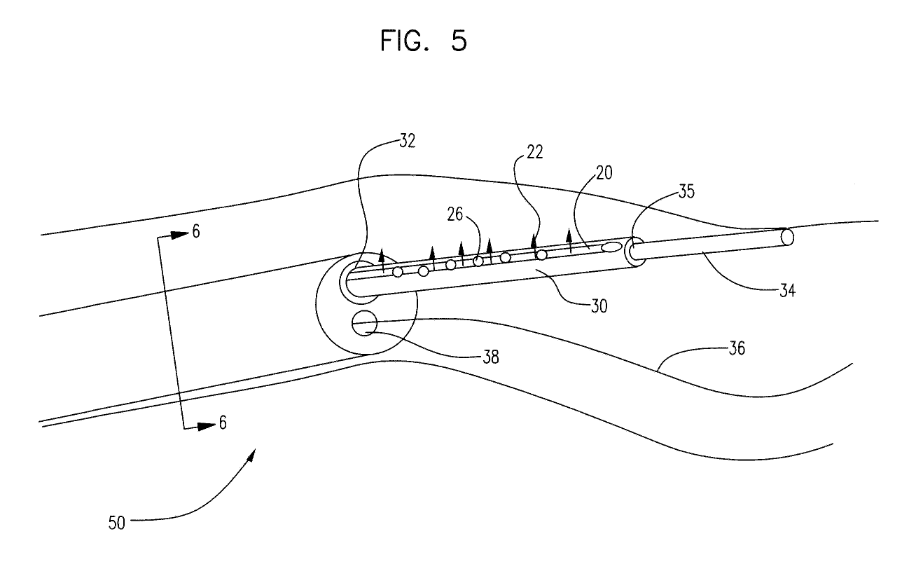

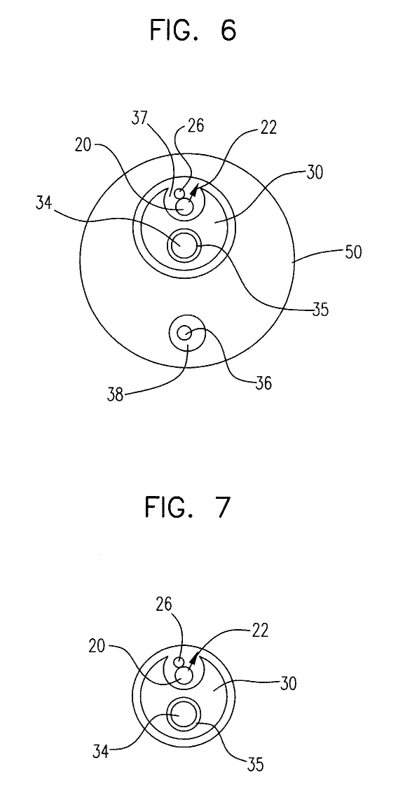

The drug delivery device may be delivered to the desired treatment site within the vessel of a patient using any method known in the art. One method is to employ a catheter delivery device. FIGS. 5, 8 and 9 are perspective views illustrating the distal end of one embodiment of a catheter delivery device shown within a patient's blood vessel. FIG. 6 is a radial cross-section taken at section 6-6 in FIG. 5 and FIG. 7 is a radial cross-section illustrating a shape memory tube.

Typically, a guidewire 36 is inserted into an incision in an artery, such as the femoral artery, and is advanced to the desired treatment site and a guiding catheter (not shown) is introduced into the patient over guidewire 36 and maneuvered to the ostium of the desired vessel. The dual-lumen delivery catheter 50, shown in the embodiment in FIG. 5, is then advanced over guidewire 36 as shown in FIG. 5 until it crosses the desired lesion, and then both are advanced through the guiding catheter to the distal end thereof. The delivery catheter 50 may then be advanced over the guidewire 34 until properly positioned for deployment of drug delivery device 10. Of course, catheters other than the dual-lumen type shown in FIG. 5 may be employed, and the delivery process may be varied as well.

In the embodiment shown in FIGS. 5-9, drug delivery device 20, which includes flexible elongate element 20, tacks or pins 22 for securing the flexible elongate element 20 to a vessel wall and drug-loaded microparticles 26 disposed on flexible elongate element 20, is embedded within a profiled wire 30, such as a shape memory wire for example a nitinol wire, hereinafter referred to as “nitinol wire 30”.

In this embodiment, nitinol wire 30, further includes a stainless steel wire 34, for maintaining nitinol wire 30 in a straight configuration. Stainless steel wire 34 is disposed within a lumen 35 of nitinol wire 30. In the embodiment shown in FIGS. 5-9, drug delivery device 10, is embedded within a channel 37 of nitinol wire 37. Wire 34 may be formed of various other rigid materials including, but not limited to, cobalt chromium titanium, as well as a rigid, hard plastic such as high density polyolefins, for example, high density polyethylene, depending on the cross dimensional shape of wire 30 as compared to wire 34.

FIG. 6 is a radial cross-section of the dual lumen catheter taken at section 6-6 in FIG. 5. Nitinol wire 30 is shown disposed within catheter lumen 32. Guidewire 36 is shown disposed within catheter lumen 38.

A radial cross-section of nitinol wire 30 is shown in FIG. 7. Drug delivery device 10 is shown disposed within channel 37 of nitinol wire 30 and straightening wire 34 is shown disposed in lumen 35 of nitinol wire 30.

Drug delivery device 10, can further be temporarily adhered to nitinol wire 30 using a readily dissolvable anionic or cationic glue such as gelatine, or with a fugitive adhesive wherein a weak bond is formed such that upon any mechanical stress, the bond is broken. The nitinol wire 30 and the straightening wire 34 can be pushed out of the catheter lumen 32 as shown in FIG. 5.

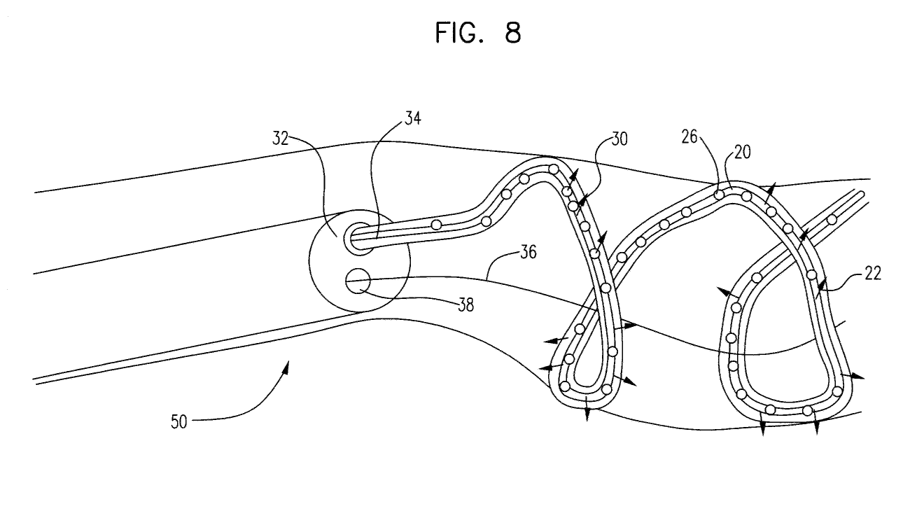

Next, the straightening wire 34 is pulled back in the direction of the arrow as shown in FIG. 8. The nitinol wire 30, free from straightening wire 34, coils into a spiral along with the drug delivery device 20 as shown in FIG. 8. As is known in the art, shape memory materials, e.g. shape memory metals, are a group of metallic materials that can return to some previously defined shape or size when subjected to the appropriate thermal procedure, i.e. they can be plastically deformed at a relatively low temperature, and upon exposure to some higher temperature, will return to their original shape. Examples of shape memory materials include, but are not limited to, copper-zinc-aluminum-nickel, copper-aluminum-nickel and nickel-titanium. For discussions of shape memory materials see for example, commonly assigned U.S. Pat. Nos. 7,163,550, 6,746,475 and 6,579,297, and U.S. Pat. No. 6,652,576, each of which is incorporated by reference herein in its entirety, and commonly assigned U.S. Patent Publication Nos. 20070123807, 20040193207 and 20020098105, each of which is incorporated by reference herein in its entirety.

Nitinol, well known in the art, can be made with an austenitic final (A_{f}) temperature above body temperature. At room temperature the nitinol wire is in its martensite phase and can be easily deformed. So, for example, the elongate deployment device can be made from nitinol, formed into a spiral or helical shape and then heat set into this shape. Thus, the spiral shape of the nitinol wire 30 can be “preset” using thermal procedures. When the nitinol wire 30 springs into its preset spiral shape, the force drives the hooks or tacks 22 of the drug delivery device 20 into the vessel wall, thereby securing the drug delivery device 20 to the vessel wall. When the nitinol wire is deployed from the catheter shaft lumen, and takes on its preset configuration, this may also be referred to herein as an expanded configuration.

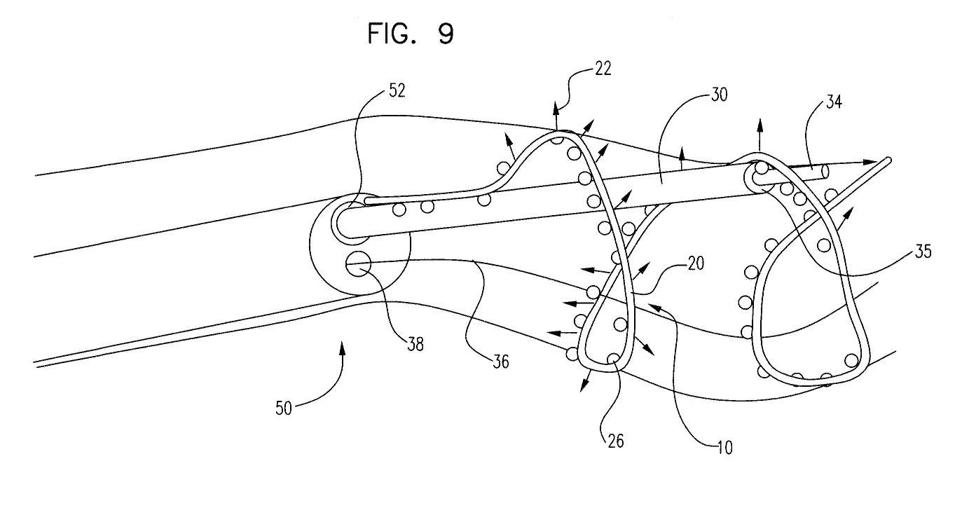

The drug delivery device 10 is released from nitinol wire 30 as shown in FIG. 9, either by dissolution of cationic or anionic adhesive within the bodily fluid, or by the force of the nitinol wire 30 as it moves into its preset spiral shape such as when a fugitive adhesive is employed. The straightening wire 34 can then be pushed back through the lumen 35 of the nitinol wire 30, thereby straightening the nitinol wire 30 so that it can be pulled back into catheter lumen 32.

The present invention can be used for the treatment of any of a variety of ailments including, for example, vascular injuries such as injuries of the coronary vasculature including obstructions, treatment of the peripheral vasculature including obstructions of the peripheral vasculature, treatment of the gastrointestinal tract such as for the treatment of Crohn's disease, treatment of the renal vasculature such as for renal insufficiency, etc.

The method may be particularly beneficial for the treatment of non-obstructive vascular lesions (NOL), for example, wherein the stenosis is less than about 60%. In this situation, it may be advantageous to delivery a therapeutic agent(s) to the affected vessel without the use of an accompanying mechanical support structure such as a stent. This can eliminate side affects that can occur with placement of such a support structure, for example, inflammation and thrombosis.

The devices, methods and compositions of delivering therapeutic agents locally and for sustained periods of time can eliminate the need for additional procedures and associated complications.

The above disclosure is intended to be illustrative and not exhaustive. This description will suggest many variations and alternatives to one of ordinary skill in this art. Those familiar with the art may recognize other equivalents to the specific embodiments described herein which equivalents are also intended to be encompassed by the claims.

1. A device adapted for implantation or insertion into a vessel of a patient for the local delivery of a therapeutic agent, the device comprising at least one flexible filament, the at least one flexible filament comprising a polymeric carrier of a therapeutic agent and anchors for securing the flexible filament to the vessel wall.

2. The device of claim 1 comprising a polymeric carrier of at least two therapeutic agents.

3. The method of claim 1 wherein said device comprises two or more flexible filaments.

4. The method of claim 3 wherein said two or more flexible filaments are intertwined.

5. The device of claim 1 wherein polymeric carrier of said therapeutic agent or mixture of therapeutic agents is a coating disposed on said at least one flexible filament.

6. The device of claim 1 wherein said polymeric carrier comprises at least one polymer comprising l-lactide, d,l-lactide, glycolide or mixtures thereof.

7. The device of claim 6 wherein said at least one flexible filament is formed from a degradable, bioresorbable metallic material.

8. The device of claim 1 wherein said polymeric carrier of a therapeutic agent comprises a plurality of polymeric microparticles disposed on said at least one flexible filament.

9. The device of claim 1 wherein said each of said plurality of said microparticles comprises a polyelectrolyte multilayer shell.

10. The device of claim 9 wherein said polyelectrolyte multilayer shell comprises an outer most layer comprising a positive charge or a negative charge, and said at least one flexible filament comprises a coating having a charge that is opposite that of the outer most layer of the multilayer shell, said plurality of microparticles are disposed on said at least one flexible filament.

11. The device of claim 8 wherein said plurality of polymeric microparticles comprises a first population of microparticles comprising a first therapeutic agent and said plurality of polymeric microparticles comprises a second population of microparticles comprising a second therapeutic agent that is different than said first therapeutic agent.

12. The device of claim 1 in combination with a delivery catheter.

13. A catheter for delivering an insertable or implantable drug delivery device to a treatment site in a vessel of a patient, the catheter comprising:
an elongate catheter shaft having an inner surface defining a lumen;
a flexible elongate drug delivery device disposed in said lumen, the flexible elongate drug delivery device comprising a carrier of a therapeutic agent and anchors for securing said drug delivery device to a vessel wall; and
an elongate deployment device having a preset expanded configuration releasably secured to said flexible elongate drug delivery device;
wherein said elongate deployment device assumes its preset expanded configuration and releases said elongate drug delivery device upon deployment in a vessel.

14. The catheter of claim 13 wherein said elongate deployment device is formed from a shape memory material.

15. The catheter of claim 13 wherein said elongate deployment device comprises a first configuration and a second configuration, said second configuration comprising a spiral shape, said elongate deployment device in its second configuration when deployed from said lumen of said catheter shaft.

16. The catheter of claim 13 further comprising an elongate straightening device for said elongate deployment device, the elongate deployment device having an inner surface defining a lumen, said elongate straightening device is disposed within said lumen of said elongate deployment device.

17. A method for deploying an insertable or implantable drug delivery device in a vessel of a patient, the method comprising:
providing a delivery catheter comprising an elongate flexible catheter shaft having an inner surface defining a lumen and having a distal portion;
providing a drug delivery device within said distal portion of said lumen of said catheter shaft, the drug delivery device comprising at least one flexible filament and a carrier of a therapeutic agent or mixtures of therapeutic agents disposed on said at least one flexible filament, and said at least one flexible filament comprising anchors for securing said drug delivery device to a wall of the vessel;
positioning the distal portion of said delivery catheter at a treatment site in the vessel; and
deploying said drug delivery device from said catheter shaft and into said vessel, wherein said anchors secure said drug delivery device to said vessel wall.

18. The method of claim 17 wherein said drug delivery device comprises two or more flexible filaments.

19. The method of claim 17 wherein said drug delivery device is releasably secured to an elongate deployment device, the elongate deployment device having a preset expanded configuration, the method comprising pushing said elongate deployment device from said lumen wherein said elongate deployment device expands, releasing said drug delivery device into said vessel.

20. The method of claim 18 wherein said elongate deployment device is an elongate shape memory deployment device.

21. The method of claim 18 wherein said elongate deployment device in said preset expanded configuration is helical.

22. The method of claim 18 wherein said elongate deployment device comprises an inner surface defining a lumen, the method comprising providing an elongate straightening device within said lumen, the elongate straightening device is engaged to said elongate deployment device.

23. The method of claim 22 wherein said elongate deployment device comprises an inner surface defining a lumen, and said elongate straightening device is disposed within said lumen of said elongate deployment device, said pushing step comprises pushing said elongate straightening device and said drug delivery device from said lumen of said catheter, said method further comprising the steps of pulling back said elongate straightening device releasing said elongate deployment device and said drug delivery device, pushing said elongate straightening device into said lumen of said elongate deployment device, and pulling said elongate straightening device and said elongate deployment device into said lumen of said catheter.

24. A method comprising:
inserting or implanting a drug delivery device into a vessel of a patient, the drug delivery device comprising at least one flexible filament and a carrier of a therapeutic agent or a mixture of therapeutic agents disposed on said at least one flexible filament, and said at least one flexible filament comprising anchors for securing said at least one flexible filament to a wall of said vessel; and
securing said elongate medical device to said vessel wall.

25. The method of claim 24 wherein said drug delivery device comprises two or more flexible filaments.

26. The method of claim 24 wherein said anchors comprise an adhesive, said adhesive effective for bonding to bodily tissue.

27. The method of claim 24 wherein said anchors comprise nails, hooks, tacks or pins.
